(19) United States
(12) Patent Application Publication
(10) Pub. No.: US 2003/0170886 A1
(43) Pub. Date: Sep. 11, 2003

(54) Method of developing an anti-protein and regulation of acellular function by administering and effective amount of the anti-protein

(76) Inventor: Lawrence F. Glaser, Fairfax Station, VA (US)

Correspondence Address: NIXON PEABODY, LLP, 8180 GREENSBORO DRIVE, SUITE 800, MCLEAN, VA, 22102, US

(21) Appl. No.: 10/329,764

(22) Filed: Dec. 27, 2002

Related U.S. Application Data
(60) Provisional application No. 60/342,357, filed on Dec. 27, 2001.

(51) Int. Cl. A61K 38/00; C12Q 1/68; C12N 5/06; C12N 5/16

(52) U.S. Cl. 435/345; 514/12; 435/6

(57) ABSTRACT

Alterations to a host cell protein form a set of specific alterations which may be deployed to then limit a trial and error process in order to arrive at an Anti-Protein targeted to a host cell protein. The invention dictates regulation of monomers, multimers, oligomeric subunits and oligomers, or proteins by changing their form and function sufficiently, to yield a new set of interaction rules which closely resemble the rules followed by naturally occurring monomers, multimers, oligomeric subunits oligomers and proteins. This Anti-Protein contains highly specific alterations, which render the ultimate presence and sufficient concentration of these compositions to yield predictably different interaction, structure and function for the cell and which incorporate the necessary coding for transcription, translation and sufficient concentrated production for these Anti-Proteins, to enable regulation of a particular cellular function, such as viral replication.

### CROSS REFERENCE TO RELATED APPLICATIONS

This application is related to U.S. Provisional Applications, under 35 U.S.C 119(e) Serial No. 60/342,357, as well as Non-Provisional Application Ser. No. 10/298,997, the entire disclosures of which are hereby incorporated by reference.

### BACKGROUND OF THE INVENTION

The primary structures observed within proteins are the linear sequence of amino acids that are bound together by peptide bonds. Change in a single amino acid in a critical area of the protein or peptide can alter biologic function as is the case in sickle cell disease and many inherited metabolic disorders. Disulfide bonds between cysteine (sulfur containing amino acid) residues of the peptide chain stabilize the protein structure. The primary structure specifies the secondary, tertiary and quaternary structure of the peptide or protein.

The secondary structure of peptides and proteins may be organized into regular structures such as an alpha helix or a pleated sheet that may repeat or the chain may organize itself randomly. The individual characteristics of the amino acid functional groups and placement of disulfide bonds determine the secondary structure. Hydrogen bonding stabilizes the secondary structure.

Genomic information does not predict post-translational modifications that most proteins undergo. After synthesis on ribosomes, proteins are cut to eliminate initiation, transit and signal sequences and simple chemical groups or complex molecules are attached. Post-translational modifications are numerous (more than 200 types have been documented), static and dynamic including phosphorylation, glycosylation and sulfonation.

Tertiary structure of proteins and peptides is the overall 3-D conformation of the complete protein. Tertiary structure relates to the steric relationship of amino acid residues that may be far removed from one another in the primary structure. Such 3-D structure is that which is most thermodynamically stable for a given environment and is often subject to change with subtle changes in environment. In vivo, folding of large multi-domain proteins occurs cotranslationally and the maturation of proteins occurs in seconds or minutes. Intracellular protein folding is regulated by cellular factors to prevent improper aggregation and facilitate translocation across membranes. The two methods for determining 3-D protein structures are nuclear magnetic resonance and x-ray crystallography.

If the functional protein consists of several subunits, the quaternary structure consists of the conformation of all the subunits bound together by electrostatic and hydrogen bonds. Multisubunit proteins are called oligomers and the various component parts are each monomers or subunits. Proteins may contain non-amino acid functional structures such as a vitamin derivative, mineral, lipid or carbohydrate.

Secondary and tertiary protein structure can be determined by two methods: X-ray crystallography and nuclear magnetic resonance. In either case the protein should be better than 95% pure for optimal results. Purification schemes vary but may include gel or column separation, dialysis, differential centrifugation, salting out, or HPLC with the choice and sequence of methods being tailored to the specific protein.

Nuclear Magnetic Resonance (NMR) spectroscopy has a distinct advantage over X-ray crystallography in that crystallization, which is often difficult and sometimes impossible, is not necessary. State of the art NMR spectroscopy considers 10,000 Dalton molecular weight (MW) structures routine, whereas 20,000 MW structures are far more challenging but have been realized, and even 30,000 MW structures have been partially determined. Technical barriers to NMR spectroscopy are diminishing and prospects are that NMR spectroscopy will eventually resolve protein structures up to 100,000 MW.

The greatest advantage of NMR spectroscopy is its facility to reveal details about specific sites of molecules without having to solve their entire structure. Another special advantage of NMR spectroscopy is its sensitivity to motions on the time scale of most chemical events, allowing direct examination of motions on the millisecond to second range and indirect studies of motions on the nanosecond to microsecond range. Modern NMR spectroscopy is particularly adept at revealing how active sites of enzymes work. One technique called Transfer Nuclear Overhauser Spectroscopy (TrNOESY) facilitates shape determination of small molecules bound to very large ones, and helps define the binding pocket of the molecule.

A modern NMR spectrometer is basically a computer controlled radio station with its antenna placed in the core of a magnet. Nuclear dipoles in the sample align in the magnetic field and can absorb energy and ‘flip’ from one orientation to another, and later flip back and readmit that energy. The stronger the field, the greater the alignment. The stronger the dipole, the greater the energy associated with the alignment. The computer directs a transmitter to pulse radio waves to the sample inside of the antenna. The sample absorbs some of the pulse, and after time readmits radio signals, which are amplified by a receiver and then stored on the computer. Nuclei in different chemical environments on molecules radiate different energies allowing investigators interpret the chemical environments utilizing software routines.

The key to the success of biological NMR spectroscopy is the hydrogen nucleus. Hydrogen is the most highly abundant element in organic molecules and has one of the strongest nuclear dipoles in nature. Using pulse sequences that take advantage of the hydrogen nucleus, investigators can map the chemical bond connectivity and the spatial orientation and distance geometry of large biomolecules. With that information and the aid of molecular modeling programs the structure of many proteins and fragments of DNA have been determined.

Despite common usage the word ‘nuclear’ does not refer to radioactive decay. Nuclear magnetism merely relates to properties of the nucleus similar to the common magnetism we are familiar with. Nuclear magnetism is many thousand times weaker per atom than the magnetism associated with electrons. Some stable isotopes have nuclear magnetism, as do some radioactive isotopes. However, nuclear magnetism is not related directly to radioactive decay. Almost all NMR spectroscopy is done with stable isotopes.

Purified protein can be induced to crystallize by various crystallization methods. The most common are batch methods and vapor diffusion. Chemically, protein in an aqueous environment is induced to associate with other protein molecules by the formation of a supersaturated solution. Specific supersaturation requirements for nucleation and growth differ from protein to protein. Supersaturation is most often achieved by the addition of precipitants such as salts or polyethylene glycol. Chemical conditions and the nature of the protein determine what conditions of pH, temperature, precipitant, and protein concentration will favor the formation of high quality crystals of sufficient size for analysis.

Crystallization conditions must be tailored to each individual protein. Reproducibility is a problem and there is much trial and error in the determination of the best crystallization conditions. An understanding of the physical and chemical properties, stability, solubility and amino acid sequence facilitate protein crystallization. Newer high-throughput crystallization methods using smaller volumes of sample require about a third the total amount of pure soluble protein necessary for conventional crystallization studies.

Once crystals of sufficient size and quality are obtained, they are mounted and snap frozen by immersion in cryogenic liquids or exposure to cryogenic gas to prevent ice lattice formation. Freezing sometimes disrupts macromolecular crystals making them useless for crystallography.

X-ray crystallography is a powerful technique whereby X-rays are directed at a crystal of protein or a derivative of the protein containing a heavy metal atom in an effort to determine secondary and tertiary structure. The rays are scattered in a pattern dependent on the electron densities in different portions of a protein. The crystal must remain supercooled during data collection. This minimizes radiation damage and backscatter, increases resolution and allows for long term storage and reuse of crystals. Images are translated into electron density maps, which superimposed on one another either manually or by specialized computer programs, allowing the scientist to construct a model of the protein. Crystallography is time consuming, expensive and requires very specialized training and equipment. However, it reveals very precise and critical structural data about amino acid orientation that is then used to understand protein interactions and design drugs in structure based drug design.

### SUMMARY OF THE INVENTION

Since a change in only one amino acid within the targeted regions of genetic coding, can indeed greatly affect the performance of a resulting protein, the specific preferred embodiment of the invention is an Anti-Protein, which in turn more predictably interacts with unaltered monomers to yield quaternary structures (multimers or oligomeric subunits) which are predictably flawed. However, the flaw in question is intended and is studied for use in biological systems. As used in this context the term flaw is completely relative. A flaw projected consistently into a resulting protein form, represents the Anti-Protein form if the subsequent interactions desired then prevail.

The primary structure of an Anti-Protein said to have Anti-Proteomic properties includes peptides and proteins, comprised of a linear sequence of amino acids that are bound together by peptide bonds. Substitution of a single amino acid, addition of amino acids or deletion of amino acids or a combination of the aforementioned permutations in the targeted amino acid sequence of a protein or peptide can alter biologic function, as is the case in sickle cell disease and many inherited metabolic disorders. Disulfide bonds between cysteine residues of the peptide chain stabilize the protein structure. The primary structure specifies the secondary, tertiary and quaternary structure of the peptide or protein. During an investigation of proetomics within a defined domain, such as those viral mediated proteins which give rise to virion synthesis and even more specifically, capsid shell formation, certain related proteins can be quickly and easily isolated and traced to the viral genome nucleic acid sequences. It is true that the genetic coding which yields a given protein, does not predict the final form of any protein translated and acted upon by other interactive proteins, enzymes, co-enzymes and the environmental characteristics encountered by said protein. But it is equally known that a given sequence of genetic coding does produce the ultimate chain of amino acids with certainty, or the protein would never translate in sufficient quantity to be of use to the overall biological system (cell) within which it has been evolved to interact.

One object of the present invention is to produce an Anti-Protein which continues to fold, imitating the protein to which it is targeted (a single, amino-acid altered protein for which this protein must displace according to significant presence, concentration or superior strength of bond) and interact with related proteins in tertiary and quaternary structure at the moment of natural monomer formation, multimer formation and oligomeric subunit formation and exhibit natural bonds, including hydrophobic, electrostatic or other. Production of the Anti-Protein then provides a method of treating or preventing viral infection or viral proliferation caused by virus insertion of a wild-type viral genome into a host cell chromosome by administering to an infected host a therapeutically effective amount of a stable, altered and reproducible wild-type viral Anti-Protein containing one or more amino acid substitutions. Upon administration, the Anti-Protein results in down regulation of at least one viral function, for example transport of the virus genome into a cell, transport of the viral genome into a cell nucleus, viral replication in the cell, viral protein synthesis and transport of virus particles from an infected cell.

Another object of the present invention is to study secondary structure(s) formation or formations and their boundaries and limitations for what was formerly a defined function for the target protein, and is a defined function for the Anti-Protein

Still another object of the present invention is to study tertiary structure(s) formation or formations and their boundaries and limitations for what was formerly a defined function for the target protein, and is a defined function for the Anti-Protein

Still another object of the present invention is to study quaternary structures formation or formations and their boundaries and limitations for what was formerly a defined function for the target protein, and is a defined function for the Anti-Protein.

Still another object of the present invention is to study monomer and multimer formations, use x-ray crystallography or electron microscopy.

Still another object of the present invention is to determine if the Anti-Protein can successfully compete with (at a sufficiently concentrated level), and displace the targeted Protein in forming monomers, multimers or oligomeric subunits which then carry a desirable trait The trait sought will be structural, mediated by enzymes, co-enzymes hydrophobic and electrostatic bonding and the substructures provided.

Still another object of the present invention is to repeat the aforementioned objects for oligomeric subunits and oligomers.

Still another object of the present invention is to prepare protocols for testing in vitro using suitable host cells and starting with simplistic viruses, moving through these steps and testing more and more complex viruses.

Still another object of the present invention is to test for toxicity, digestion, immunological or antigenic, other forms of molecular interference, promotion or interaction.

### DETAILED DESCRIPTION OF THE INVENTION

The monomers, multimers and oligomeric subunits formed either inhibit oligomer formation due to three dimensional changes in the precursor structures or by way of changes in bonding affinity reduction or increase at given points, or combinations of the two desirable effects.

The ideal Anti-Protein follows the natural course of the target protein. The Anti-Proteins described herein fold, form secondary, tertiary, and quaternary structures. In the case of tertiary and quaternary structures, the monomers, multimers or oligomeric subunits that form inclusive of this Anti-Protein , i.e., oligomer containing said precursor structures, is equally as prolific if not more prolific, digestible, non-interfering and immunosilent as the unaltered target protein. As these desirable features approach the logical maximum, the Anti-Protein is, by definition here, the most preferred embodiment. Monomers, multimers, oligomeric subunits and oligomers can be termed Anti-Monomer-1, Anti-Multimer-1, Anti-Oligomeric Subunit-1 or Anti-Oligomer-1, where the suffix indicates for each, how many Anti-Protein/Protein substitutions have been successfully incorporated and, in the case of the Oligomer or Oligomeric Subunits, how many Monomers or Multimers bear at least one Anti-Protein substitution. Some viral capsids, as an example, may produce several classes of monomer that ultimately combine in substructures to inevitably form one oligomer Therefore, we provide for an initial lexigraphy to guide the researcher.

The number of target proteins, monomers, multimers, oligomeric subunits and oligomers of interest in any particular case is dependent upon the targeted system. For example, the viral genome typically (but variably) code for capsids, nucleocapsids, matrix proteins, enzymes, hoist proteins, other viral membranes, and glycoproteins. Essentially all viral proteome components can become targets. The lower the concentration of a given proteome component in the natural environment of the proteome in question, during the mode a researcher wishes to down-regulate or completely shut down, the greater the expedience at which a therapeutic effect will be observed. This teaching can be applied by one skilled in the art to regulate many processes inclusive of viral mediated processes and as well as exclusive (non-viral) processes which appear within living cells, organs, biological systems, bacterium, fungi, phage, viriod, plasmoid or any protein specific process one wishes to regulate and control.

In one embodiment of the present invention, in a virus genome, 7 capsid forming protein domains are discovered through techniques known to those skilled in the art (e.g. in situ probing, crystallographic verification). For purposes of this illustration, these domains are labeled as glp, g2p, g3p, g4p, g5p, g6p and g7p. Upon acquisition of the three dimensional structure of the oligomer, which represents the viral capsid shell, it is clearly determinable that g3p represents the most prolific and present protein, and g3p happens to, in this example, consist of the shortest amino acid sequence of only 7 amino acids which gives rise to the protein in question. Amino acids are substituted by altering the nucleotides in the viral DNA.

In vitro experiments and observation of the performance of several modified g3p+ proteins using a suitable host producer cell line and only purified and greatly amplified clonal DNA from the modified viral genome, bearing only the g3p+ sequence alteration(s) within the natural target virus remaining sequence data, yields data which indicates that the 7 proteins are now incapable of forming the shell capsid oligomer form. The common denominator is the presence of mostly g3p+5G/A Anti-Monomers (in this example, the 4^{th} substitution attempt which used GAATACACCTTAGGATAGATA as the primary sequence and found that substituting base 5 “A” with “G” yielded the following observation. The lexicon use tells us this is an Anti-Protein, the ₅ amino acid was substituted, G was substituted for A). Thus, no full virion capsid shell formation remains in this experiment. This could be verified by Electron Microscopy and over an elapsed period, through electron microscopy filming techniques.

Thereafter, when tested again in identical fashion, but in competition with wild-type variants, at a certain presence level for the g3p+5G/A translating genome (g3p+5G/A-Anti-Monomer-1), again no valid capsid shell formation would be visible in vitro. Perhaps Anti-Multimers, Anti-Oligomeric Subunits and Anti-Oligomers would form. Given that the prior oligomer was a sphere or Isocyhedron, a possibility would include a warped anti-multimer, forming a chain of bow-tie formations. Said formation would become digested by natural cellular cytoplasmic cycling and digestion enzyme function, thus many of the fundamental and essential building blocks could be efficiently recovered without much loss of energy within the cell. (Energy conservation is important, but virion synthesis down regulation is the paramount goal).

Over-expression of g3p+5G/A, would be deemed a potential improvement, as the over-expression is easily mitigated either through repeats of the sequence which gives rise to the g3p+5G/A Anti-Protein, along with appropriate flanking sequences to mitigate transportation and natural cleaving, or promotion enhancement or other techniques known to those skilled in the art which is capable of yielding more efficient and acceleratory expression of mRNA, transport, translation processes yielding final cleaving into functional protein(s), combinations of these techniques and other techniques. Provided the g3p+5G/A Anti-Protein and the g3p+5G/A-Anti-Monomer-1 are stable and non-toxic, and do not demonstrate an issue for cellular recycling, the overall experiment will be successful.

We will have demonstrated a safe, reliable and easily replicable means to down-regulate a targeted viral proteome and greatly curtail or eliminate viral capsid formation within a given cell (reduce or eliminate virion production). Infection of each cell is required with a virion composition containing a viral genome which transcribes and translates as defined herein.

Hence the concept of the Anti-Protein, Anti-Proteomics and the fundamental teachings on how it is possible to study a genome, the related proteome and the logical protein, enzyme, monomer, multimer, oligomeric subunit and oligomer structural output and the process of isolation of Anti-Protein targets from within the natural array of proteins within the given proteome are herein disclosed.

Genomic information does not predict post-translational modifications that most proteins undergo, however, changes to the genomic information will yield changes in post-translational proteomic fundamentals, such as the anticipated structure, its environmental stability and its other characteristics as compared to the original unaltered genomic information and the naturally translated protein(s). After synthesis at suitable ribosomes, proteins are cut to eliminate initiation, transit and signal sequences and simple chemical groups or complex molecules may be attached. Anti-Proteins must follow the same synthesis and post-synthesis interactions and pathways. Post-translational modifications are numerous (more than 200 types have been documented), static and dynamic including phosphorylation, glycosylation and sulfonation. Anti-Proteins can make use of any post-translational modification. The clear intent of the present invention is to displace a targeted protein, typically through concentrated presence and frequent substituted interaction with related proteins, then forming Anti-Monomers, Anti-Multimers, Anti-Oligomeric Subunits then forming Anti-Oligomers and disrupting at the physical level, a given proteome mediated form causing disruption of a natural cycle.

Tertiary structure of Anti-Proteins and peptides is the overall 3-D conformation of the complete Anti-Protein. Tertiary structure considers the steric relationship of amino acid residues that may be far removed from one another in the primary structure. Such 3-D structure is that which is most thermodynamically stable for a given environment and is often subject to change with subtle changes in environment. In vivo, folding of large multidomain Anti-Proteins will occur cotranslationally and the maturation of Anti-Proteins occurs in seconds or minutes. Intracellular Anti-Protein folding is regulated by cellular factors to prevent improper aggregation and facilitate translocation across membranes. Some Anti-Proteins may be selected for the membrane translocation property or the opposite property, to not translocate through a membrane. Two methods for determining 3-D Anti-Protein structures include nuclear magnetic resonance and x-ray crystallography.

If the functional Anti-Protein consists of several subunits, the quaternary structure consists of the conformation of all the subunits bound together by electrostatic and hydrogen bonds. Multisubunit Anti-Proteins combined into their final form (typically the largest molecular weighted form) are called Anti-Oligomers and the various component parts are each Anti-Monomers, Anti-Multimers, Anti-Oligomeric Subunits or simply “Anti-Subunits”. Anti-Proteins may contain non-amino acid functional structures such as a vitamin derivative, mineral, lipid or carbohydrate. Ultimately, the idea is to substitute an anti-protein for a protein, with predictable and reliable frequency, which then follows the precise pathway of the target protein but causes only the effects the researcher desires. If a viral genome forms capsids, the effect is to disrupt normal monomer, multimer, oligomeric subunit and oligomer formation, replacing them with complementary Anti-Monomers, Anti-Multimers, Anti-Oligomeric Subunits and Anti-Oligomers which no longer yield capsid shells. This is accomplished through the successful synthesis of Anti-Monomers, Anti-Multimers, Anti-Oligomeric Subunits and Anti-Oligomers which are compatible with (for example and in one preferred embodiment) the human cell, while remaining incompatible with virion synthesis due to the incorporation of an unsuitable Anti-Oligomeric structure, e.g. a flawed capsid which does not bud or escape, cannot hoist a genome, does not host matrix formation or nucleocapsid placement or formation, is non-toxic, easily digested and immunosilent. In another embodiment, Anti-Proteins are made for any virus, subunit of a virus or cell, any protein, and any function of a cell or protein. This is intended to span the entire universe of nucleic acid based pathogen(s), cells and life forms.

This teaching is intended to complement another method of treating infection, termed “TheraVirus” which is subject of Non-Provisional Application Ser. No. 10/298,997, the entire disclosures of which are hereby incorporated by reference. These teachings can be taken a step further. Gene therapy continues to be a very new, early stage domain within which there is plenty of opportunity to pioneer and to stake out certain new areas of research. These teachings lead one to a greater conclusion. It is believed these teachings indicate HIV-1 could be shut down with a lineage of virions (TheraVirus) which express a single (deliberate) defective capsid forming shell protein, and otherwise maintain replication incompetence and block HIV−1 replication further through occupation of integrated positions within suitable human cells(at palindromes, within chromosomes). HIV−1 in this form (TheraVirus) offers a perfect platform for delivering human gene therapy in the cell types which HIV−1 favors. Altered surface glycoproteins, through genome modification, to favor other human cell types, will enable the TheraVirus platform to continue to provide a useful function. HIV−1 is a product of evolution and its overall specificity may well serve to prove that the definitive TheraVirus structure can be safely maintained and used successfully as a human gene therapy platform (immunosilent) vector. HIV−1 offers a unique inherit feature seemingly adaptable as a universal vector, as it is capable of infecting a resting cell or immature cell. A review of the TheraVirus disclosure of the above related application, one quickly sees the overall molecular specificity of the whole virion is maintained. Additionally, rather than attenuate the viral genome to a high degree, these teachings take an opposite approach.

While leaving the viral genome mostly intact, change a minimized set of codings into a genome which renders the overall form capable of certain cyclic functions of the virion and incapable of replication. At the same time, within the same composition, we deliver limited expression capacity which is not easily eliminated through mutation. Then, express certain mRNAs which ultimately produce a protein, deemed by these teachings to be an Anti-Protein, to yield Anti-Monomers, Anti-Multimers, Anti-Oligomeric Subunits Anti-Oligomers and ultimately, greatly disable capsid formation for any pre-infected targeted host cell by down regulating or completely shutting down all capsid shell formation, budding and transport. Also intriguing is the prospect that a TheraVirus will not disrupt energy consumption cycles and fundamental building block utilization in the cells it infects, because it transcribes and translates so very few actual mRNAs. If its presence blocks subsequent wild-type HIV−1 infection and at the same time, for those few HIV−1 variants that integrate within the same cell as TheraVirus, it is seen that the virion production is slim to none, the anticipated overall effect of TheraVirus would be to literally cure a host of the HIV−1 infection. The use of the term cure represents two possibilities. Safe control of HIV−1 wherein the host is a carrier for life or, in the alternative, the further observation that long term use of TheraVirus leads to complete eradication of natural, wild-type HIV−1 inclusive of its complete elimination. The possibility of complete elimination exists through extreme levels of the TheraVirus technique, used in experimentation to demonstrate this particular function. If HIV−1 is essentially safe in high concentration, as is observed in HIV−1+ patients, its TheraVirus antithesis is believed to be safe to a much a greater magnitude. The reason resides in the location of promotor and terminator sequences and other built in alterations, as defined in the related TheraVirus application above.

In yet another embodiment of the invention, capsids which deliberately leak (imperfect spheres, or punctured spheres) can be engineered. These defective capsids would bud from the co-infected cell (in this example, a cell infected with natural HIV−1 and a special form of TheraVirus). In turn, this will serve to improve immune system detection (TREC diversity and Surface Receptor Specificity) to prompt T-Cell and Lymphocyte negative and positive selection processes, maturation processes, thymopoesis, and train those immune system components which can attack infected cells to better police and remove natural pathogenic HIV−1 infected cells from the overall host.

Vaccination is a process discovered, at least in part, through the introduction of human virus into animals. Study of the animal's blood system components, yielded the change in blood system componentry, “before” versus “after”. The layers (centrifuged) which appeared in the animal's blood, post infection and post successful immune system provocation, yielded viral components with new (unknown) antigen attached. These components, when introduced into humans prior to infection or even subsequent to infection, assisted the immune system in passing on the structural molecular elements of the animal component (killed virus and antigen attached) as “information” upon which the human immune system could not only incorporate, but memorize the teachings of this “killed virus with antigen” and retain stable resistance to what otherwise represents a fatal, pathogenic virus. Experiments along these lines using HIV have utterly failed. This inventor believes the reason for the failure is the fact that HIV is stable and immunosilent in its virion form. Viral genomes which code for anything less, are not successful or prolific in virion production. Hence, the applique of HIV (Human forms) to animals to attempt a repeat of history (e.g. successful production of killed virus with antigen) have failed. However, if the Anti-Protein and TheraVirus process can deliver a host of leaky virions (e.g. a consistent pattern of sizable access to the virion interior core), it is believed these leaky virions, produced enmasse, may well spawn the desirous “killed virus with new, unknown antigen attached” from a suitable horse, pig, hamster, mouse, rat or other suitable source (vaccination experimental model) leading to a bona-fide HIV vaccination technique. The key may well be the production of HIV virions which are homogeneously formed, each virion bearing the same Anti-Protein mediated “flaw” which removes the immunosilent feature of HIV, exposing interior glycoprotein(s), matrix protein(s) and other viral proteins to the immune system, for detection and potential antigen generation. These teachings suggest at least three approaches to HIV using TheraVirus. Each approach is embodied in the form of a virion. One approach is a blocker TheraVirus virion, which uses no Anti-Protein (occupy all palindrome locations). Another approach is a TheraVirus virion which blocks and also includes promotion of an Anti-Protein yielding deliberately defective capsids which do bud and are leaky or internally exposed (probably will cause a side effect and will be introduced very gradually, e.g. immune-response, fever, discomfort). Lastly, I suggest production of a TheraVirus virion which blocks, and also includes promotion of an Anti-Protein which completely blocks all virion capsid production. One skilled in the art can now see numerous variations on this theme, which can include blocking virions for virus' which integrate their DNA, or non-blocking virions which simply compete with the pathogenic viral RNA and DNA but either produce or selectively over-produce suggested Anti-Proteins. If TheraVirus is tolerated perpetually and if virion capsid production can be halted, or if deliberately leaky virions are produced which train the immune system through positive selection processes to clear infected cells, there is great hope for a new therapeutic modality using this approach.

What is claimed is:

1. A method of treating or preventing viral infection or viral proliferation caused by virus insertion of a wild-type viral genome into a host cell chromosome comprising the step of administering to a host a therapeutically effective titre of an stable, altered and reproducible wild-type viral anti-protein containing one or more amino acid substitutions which upon administration causes down regulation of at least one viral mediated function selected from the group consisting of transport of the viral genome into a cell, transport of a viral genome into a cell nucleus, viral genome replication in the cell, viral protein synthesis and transport of virus particles from an infected host cell.

2. The method according to claim 1, wherein administration of the viral anti-protein causes down regulation of viral capsid formation.

3. The method according to claim 1, wherein administration of the viral anti-protein additionally causes down regulation of a non-viral cellular process of the infected host cell.

4. A method of regulating or controlling a cellular function of a host cell comprising the step of administering to a host a therapeutically effective titre of an stable, altered and reproducible anti-protein containing one or more amino acid substitutions which upon administration of the anti-protein causes regulation or control of at least one cellular function.
